(19) United States
(12) Patent Application Publication
(10) Pub. No.: US 2017/0065327 A1
(43) Pub. Date: Mar. 9, 2017

(54) METHODS AND MATERIALS FOR TREATING ELEVATED SYMPATHETIC NERVE ACTIVITY CONDITIONS

(71) Applicant: Mayo Foundation for Medical Education and Research, Rochester, MN (US)

(72) Inventors: Michael J. Joyner, Rochester, MN (US); Timothy B. Curry, Rochester, MN (US); Jill N. Barnes, Madison, WI (US)

(73) Assignee: Mayo Foundation for Medical Education and Research, Rochester, MN (US)

(21) Appl. No.: 15/120,315

(22) PCT Filed: Apr. 1, 2015
(86) PCT No.: PCT/US15/23900

§ 371 Date: Aug. 19, 2016

Related U.S. Application Data
(60) Provisional application No. 61/973,380, filed on Apr. 1, 2014.

(51) Int. Cl. A61B 18/12; A61N 1/05; A61K 31/216; A61K 31/4188; A61K 31/4164; A61M 5/142; A61N 1/36

(52) U.S. Cl. CPC A61B 18/12; A61M 5/14276; A61N 1/0551; A61N 1/36057; A61K 31/4188; A61K 31/4164; A61K 31/216; A61B 2018/00434

(57) ABSTRACT

This document provides methods and materials involved in treating hypertension (e.g., age-associated hypertension, resistant hypertension, or chronic refractory hypertension), heart failure (e.g., congestive heart failure), or kidney disease (e.g., chronic kidney disease). For example, methods and materials involved in administering one or more sympatholytic agents to a patient to identify the patient as having an elevated baseline level of sympathetic nerve activity and treating the identified patient with a sympatholytic therapy to reduce the symptoms of hypertension (e.g., resistant hypertension), heart failure (e.g., congestive heart failure), or kidney disease (e.g., chronic kidney disease) are provided.

### CROSS REFERENCE TO RELATED APPLICATIONS

This application claims the benefit of U.S. Provisional Ser. No. 61/973,380 filed Apr. 1, 2014. This disclosure of the prior application is considered part of (and is incorporated by reference in) the disclosure of this application.

### STATEMENT AS TO FEDERALLY SPONSORED RESEARCH

This invention was made with government support under HL083947 awarded by the National Institutes of Health. The government has certain rights in the invention.

### BACKGROUND

1. Technical Field

This document relates to methods and materials involved in treating conditions having an elevated level of sympathetic nerve activity such as hypertension (e.g., resistant hypertension), heart failure (e.g., congestive heart failure), or kidney disease (e.g., chronic kidney disease) accompanied with elevated level of sympathetic nerve activity. For example, this document relates to methods and materials involved in administering a sympatholytic agent (e.g., a ganglionic blocking agent) to a patient to identify the patient as having an elevated baseline level of sympathetic nerve activity. Once identified, the patient is treated with a sympatholytic therapy (e.g., a neuroablation technique, an implantable device designed to deliver a sympatholytic therapy, or both).

2. Background Information

Medical conditions such as heart failure, hypertension, and peripheral vascular disease are major public health concerns. For example, nearly six million Americans currently live with heart failure and about 670,000 to 700,000 new diagnoses are made each year in the U.S. With new diagnostic technologies, improved survival after myocardial infarction, and the increasing age of the population, it is anticipated that this upward trend will continue. This includes both classical systolic heart failure as well the growing population of diastolic heart failure or heart failure with preserved ejection fraction (HFpEF). Data from the National Health and Nutrition Examination Survey, collected from 1988 through 1991, suggested that 24% of the U.S. adult population had hypertension with numbers that may be approaching 30% today. The term “peripheral vascular disease” includes peripheral arterial disease as well as venous insufficiency. It is estimated that 5% of individuals over the age of 50 years have peripheral arterial disease, although the incidence of undiagnosed peripheral arterial disease may be as high as 30%. Venous insufficiency, a form of peripheral vascular disease, has an incidence of about 2 to 5% in the U.S. population. Trends in obesity, diabetes, and inactivity are likely to increase the incidence of these major chronic illnesses.

### SUMMARY

This document provides methods and materials involved in treating conditions having an elevated level of sympathetic nerve activity such as hypertension (e.g., age-associated hypertension, resistant hypertension, or chronic refractory hypertension), heart failure (e.g., congestive heart failure), or kidney disease (e.g., chronic kidney disease) accompanied with elevated level of sympathetic nerve activity. For example, this document provides methods and materials involved in administering one or more sympatholytic agents (e.g., a combination of phentolamine followed by esmolol or a ganglionic blocking agent such as trimethaphan camsylate) to a patient to identify the patient as having an elevated baseline level of sympathetic nerve activity. After identifying the patient as having an elevated baseline level of sympathetic nerve activity, the patient is treated with a sympatholytic therapy (e.g., a neuroablation technique, an implantable device designed to deliver a sympatholytic therapy, or both) to reduce the symptoms of hypertension (e.g., resistant hypertension), heart failure (e.g., congestive heart failure), or kidney disease (e.g., chronic kidney disease).

As described herein, measuring blood pressure before and during (or before, during, and after, or before and after) administration of a sympatholytic agent (e.g., a ganglionic blocking agent such as trimethaphan camsylate) to a patient can allow clinicians to identify those patients who have an elevated baseline level of sympathetic nerve activity and who are likely to respond favorably to a sympatholytic therapy such as a neuroablation technique, a therapy that includes implanting an implantable device designed to deliver a sympatholytic therapy, or both. In some cases, a combination of phentolamine followed by esmolol can be used in place of a ganglionic blocking agent.

After identifying the patient as having an elevated baseline level of sympathetic nerve activity, the patient is treated with a sympatholytic therapy (e.g., a neuroablation technique, an implantable device designed to deliver a sympatholytic therapy, or both). For example, electrical neuroablation, chemical neuroablation, or other types of techniques can be used to block or reduce sympathetic nerve activity to reduce the symptoms of hypertension (e.g., resistant hypertension), heart failure (e.g., congestive heart failure), or kidney disease (e.g., chronic kidney disease). In some cases, renal nerve ablation (e.g., renal denervation by radio frequency ablation), baroreceptor stimulation, and/or manipulation of afferent feedback from the carotid body can be used to block or reduce sympathetic nerve activity.

Blocking or reducing sympathetic nerve activity can reduce symptoms, disrupt pathophysiology, and improve health status in patients suffering from various medical conditions associated with an elevated sympathetic nerve activity. Any appropriate chemical technique, electrical technique, or combination thereof can be used to reduce or block sympathetic nerve activity in a manner that results in a clinical improvement for a patient identified as having elevated sympathetic nerve activity and suffering from a medical condition such as heart failure, CHF, heart failure disordered breathing, dyspnea, peripheral vascular disease (e.g., peripheral arterial disease or venous insufficiency), hypertension (e.g., age-associated hypertension, resistant hypertension, or chronic refractory hypertension), chronic obstructive pulmonary disease (COPD), sleep apnea, or diabetes (e.g., diabetes mellitus type II). For example, an implantable electrode device designed to deliver electrical pulses capable of reducing or blocking sympathetic nerve activity can be positioned within a mammal (e.g., a human) with refractory hypertension such that the electrode device reduces or blocks sympathetic nerve activity from the spinal cord by greater than 25 percent (e.g., from 25 to 100 percent, from 25 to 95 percent, from 25 to 90 percent, from 25 to 75 percent, from 25 to 50 percent, from 35 to 95 percent, from 40 to 80 percent, or from 50 to 95 percent).

In some cases, a medical condition associated with elevated sympathetic nerve activity such as heart failure disordered breathing, dyspnea, peripheral vascular disease (e.g., peripheral arterial disease or venous insufficiency), COPD, sleep apnea, or diabetes (e.g., diabetes mellitus type II) can be treated as described herein. For example, a person with sleep apnea can be administered one or more sympatholytic agents (e.g., phentolamine followed by esmolol or a ganglionic blocking agent such as trimethaphan camsylate) to identify that person as having an elevated baseline level of sympathetic nerve activity. After identifying that patient as having an elevated baseline level of sympathetic nerve activity, the patient is treated with a sympatholytic therapy (e.g., a neuroablation technique, an implantable device designed to deliver a sympatholytic therapy, or both) to reduce one or more symptoms of sleep apnea.

In general, one aspect of this document features a method for treating hypertension. The method comprises, or consists essentially of, (a) administering one or more sympatholytic agents to a mammal having hypertension, (b) detecting a greater than 20 mmHg reduction in blood pressure after administration of the one or more sympatholytic agents to the mammal, and (c) ablating or stimulating an efferent or afferent nerve within the mammal or implanting a pump configured to deliver a sympatholytic agent to the efferent or afferent nerve, wherein a symptom of the hypertension is reduced following the step (c). The mammal can be a human. The hypertension can be resistant hypertension. The step (a) can comprise administering trimethaphan camsylate as the one or more sympatholytic agents. The step (a) can comprise administering phentolamine and esmolol as the one or more sympatholytic agents. The step (c) can comprise ablating the efferent or afferent nerve within the mammal. The step (c) can comprise implanting the pump configured to deliver the sympatholytic agent to the efferent or afferent nerve. The method can comprise ablating a renal nerve. The method can comprise ablating a renal nerve by applying radiofrequency ablation to the renal nerve.

In another aspect, this document features a method for treating heart failure. The method comprises, or consists essentially of, (a) administering one or more sympatholytic agents to a mammal with heart failure, (b) detecting a greater than 20 mmHg reduction in blood pressure after administration of the one or more sympatholytic agents to the mammal, and (c) ablating or stimulating an efferent or afferent nerve within the mammal or implanting a pump configured to deliver a sympatholytic agent to the efferent or afferent nerve, wherein a symptom of the heart failure is reduced following the step (c). The mammal can be a human. The heart failure can be congestive heart failure. The step (a) can comprise administering trimethaphan camsylate as the one or more sympatholytic agents. The step (a) can comprise administering phentolamine and esmolol as the one or more sympatholytic agents. The step (c) can comprise ablating the efferent or afferent nerve within the mammal. The step (c) can comprise implanting the pump configured to deliver the sympatholytic agent to the efferent or afferent nerve. The method can comprise ablating a renal nerve. The method can comprise ablating a renal nerve by applying radiofrequency ablation to the renal nerve.

In another aspect, this document features a method for treating kidney disease. The method comprises, or consists essentially of, (a) administering one or more sympatholytic agents to a mammal with kidney disease, (b) detecting a greater than 20 mmHg reduction in blood pressure after administration of the one or more sympatholytic agents to the mammal, and (c) ablating or stimulating an efferent or afferent nerve within the mammal or implanting a pump configured to deliver a sympatholytic agent to the efferent or afferent nerve, wherein a symptom of the kidney disease is reduced following the step (c). The mammal can be a human. The kidney disease can be chronic kidney disease. The step (a) can comprise administering trimethaphan camsylate as the one or more sympatholytic agents. The step (a) can comprise administering phentolamine and esmolol as the one or more sympatholytic agents. The step (c) can comprise ablating the efferent or afferent nerve within the mammal. The step (c) can comprise implanting the pump configured to deliver the sympatholytic agent to the efferent or afferent nerve. The method can comprise ablating a renal nerve. The method can comprise ablating a renal nerve by applying radiofrequency ablation to the renal nerve.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph plotting muscle sympathetic nerve activity (MSNA; bursts/min) as measured using a microneurography technique for 24 humans. The dose indicates the amount of trimethaphan in mg/min it took to abolish the sympathetic nerve bursts. The MSNA bursts per minute is the amount of sympathetic bursts that occurred at baseline, prior to drug infusion.

FIG. 2 is a graph plotting the same dose of trimethaphan in mg/min compared to the difference in mean arterial pressure (MAP) between baseline and at the final trimethaphan dose.

### DETAILED DESCRIPTION

This document provides methods and materials involved in treating hypertension (e.g., age-associated hypertension, resistant hypertension, or chronic refractory hypertension), heart failure (e.g., congestive heart failure), or kidney disease (e.g., chronic kidney disease) associated with an elevated level of sympathetic nerve activity. For example, this document provides methods and materials involved in administering one or more sympatholytic agents (e.g., a combination of phentolamine followed by esmolol or a ganglionic blocking agent such as trimethaphan camsylate) to a patient to identify the patient as having an elevated baseline level of sympathetic nerve activity. After identifying the patient as having an elevated baseline level of sympathetic nerve activity, the patient is treated with a sympatholytic therapy (e.g., a neuroablation technique, an implantable device designed to deliver a sympatholytic therapy, or both) to reduce the symptoms of hypertension (e.g., resistant hypertension), heart failure (e.g., congestive heart failure), or kidney disease (e.g., chronic kidney disease).

As described herein, one or more sympatholytic agents can be administered to mammals (e.g., humans) and blood pressure and/or sympathetic nerve activity can be monitored to identify those mammals that have an elevated baseline level of sympathetic nerve activity. Responses (e.g., reduced blood pressure and/or reduced sympathetic nerve activity) to administration of one or more sympatholytic agents can occur between about 3 minutes and about 45 minutes (e.g., between about 5 minutes and about 45 minutes, between about 6 minutes and about 35 minutes, or between about 6 minutes and about 25 minutes) of administration. Examples of sympatholytic agents that can be administered to a mammal include, without limitation, ganglionic blocking agents such as trimethaphan camsylate, hexamethonium, or pentalenium. In some cases, a non-specific α-adrenergic receptor blocker such as phentolamine, phenoxybezamine, or tolazoline or an α1-adrenergic receptor blocker such as a alfuzosin, prazosin, doxazosin, tamsulosin, terazosin, or silodosin can be administered in combination with a β-adrenergic receptor blocker such as esmolol, metoprolol, propranology, or labetalol. In some cases, a non-specific α-adrenergic receptor blocker or an al-adrenergic receptor blocker can be administered before or after administration of a β-adrenergic receptor blocker. Other examples of sympatholytic agents that can be administered to a mammal and used as described herein include, without limitation, dexmetatomadine, guanethadine, and clonidine.

Any appropriate method can be used to determine if the mammal (e.g., human) receiving the one or more sympatholytic agents has an elevated baseline level of sympathetic nerve activity. For example, blood pressure can be monitored before and during administration of the one or more sympatholytic agents to determine if the sympatholytic agents resulted in a reduction in blood pressure that is greater than 20 mmHg (e.g., a greater than 20, 25, 30, 35, 40, or 45 mmHg reduction in blood pressure). Those mammals having a reduction in blood pressure greater than 20 mmHg (e.g., a greater than 20, 25, 30, 35, 40, or 45 mmHg reduction in blood pressure) can be classified as having high sympathetic nerve activity. Those mammals having a reduction in blood pressure that is less than 20 mmHg (e.g., a less than 20, 18, 15, 10, or 5 mmHg reduction in blood pressure) can be classified as having low sympathetic nerve activity. In some cases, blood pressure can be assessed by measuring the beat-to-beat measurements of arterial pressure. Other examples for assessing blood pressure include, without limitation, arterial catheters and standard blood pressure cuffs. In some cases, blood pressure can be monitored during, after, or both during and after administration of the one or more sympatholytic agents to determine if the sympatholytic agents resulted in a particular reduction in blood pressure.

In some cases, sympathetic nerve activity (e.g., muscle sympathetic nerve activity) can be monitored before and during administration of the one or more sympatholytic agents to determine if the sympatholytic agents resulted in a reduction in the number of bursts per minute in muscle sympathetic nerve activity that is greater than 10 bursts per minute (e.g., a greater than 10, 15, 20, 25, or 30 bursts per minute reduction in muscle sympathetic nerve activity). Those mammals having a reduction in the number of bursts per minute in muscle sympathetic nerve activity that is greater than 10 bursts per minute (e.g., a greater than 10, 15, 20, 25, or 30 bursts per minute reduction in muscle sympathetic nerve activity) can be classified as having high sympathetic nerve activity. Those mammals having a reduction in the number of bursts per minute in muscle sympathetic nerve activity that is less than 10 bursts per minute (e.g., a less than 10, 8, 5, or 3 bursts per minute reduction in muscle sympathetic nerve activity) can be classified as having low sympathetic nerve activity.

In some cases, sympathetic nerve activity (e.g., muscle sympathetic nerve activity) can be monitored before and during administration of the one or more sympatholytic agents to determine if the sympatholytic agents resulted in a 25 percent or more reduction in muscle sympathetic nerve activity (e.g., a greater than 25, 30, 35, 40, 45, 50, 60, 70, or 80 percent reduction in muscle sympathetic nerve activity). Those mammals having a 25 percent or more reduction in muscle sympathetic nerve activity (e.g., a greater than 25, 30, 35, 40, 45, 50, 60, 70, or 80 percent reduction in muscle sympathetic nerve activity) can be classified as having high sympathetic nerve activity. Those mammals having a less than 25 percent reduction in muscle sympathetic nerve activity (e.g., a less than 25, 20, 15, 10, or 5 percent reduction in muscle sympathetic nerve activity) can be classified as having low sympathetic nerve activity.

In some cases, sympathetic nerve activity can be assessed by measuring muscle sympathetic nerve activity using microneurography techniques. In some cases, sympathetic nerve activity (e.g., muscle sympathetic nerve activity) can be monitored during, after, or both during and after administration of the one or more sympatholytic agents to determine if the sympatholytic agents resulted in a particular reduction in sympathetic nerve activity (e.g., muscle sympathetic nerve activity).

After identifying the mammal (e.g., human) as having high sympathetic nerve activity, the mammal is treated with a sympatholytic therapy (e.g., a neuroablation technique, an implantable device designed to deliver a sympatholytic therapy, or both). For example, electrical neuroablation, chemical neuroablation, or other types of techniques can be used to block or reduce sympathetic nerve activity to reduce the symptoms of hypertension (e.g., resistant hypertension), heart failure (e.g., congestive heart failure), or kidney disease (e.g., chronic kidney disease). In some cases, renal nerve ablation (e.g., renal denervation by radio frequency ablation), baroreceptor stimulation, and/or manipulation of afferent feedback from the carotid body can be used to block or reduce sympathetic nerve activity.

In some cases, a sympatholytic therapy can be used to reduce or block sympathetic nerve activity by targeting one or more of locations within a mammal. For example, when treating a mammal (e.g., a human) with resistant hypertension that was identified as having high sympathetic nerve activity as described herein, that mammal can undergo a neuroablation or modulation technique such as renal denervation, splanchnic denervation, carotid body denervation, carotid sinus nerve stimulation, spinal cord afferent block, other visceral efferent or afferent denervation, or a combination thereof. In some cases, when treating a mammal (e.g., a human) with congestive heart failure, heart failure respiration, dyspnea, chronic kidney disease, peripheral vascular disease, COPD, or sleep apnea, that mammal can undergo a neuroablation or modulation technique such as renal denervation, splanchnic denervation, carotid body denervation, carotid sinus nerve stimulation, spinal cord afferent block, other visceral efferent or afferent denervation, or a combination thereof.

Any appropriate electrical and/or chemical technique can be used to reduce or block efferent sympathetic nerve activity. For example, one or more electrode devices, one or more drug pump devices, or a combination one or more electrode devices and one or more drug pump devices can be used to reduce or block sympathetic nerve activity. In some cases, a mild cooling device, a vibration device, or an ultrasound device can be used to block sympathetic nerve activity (e.g., to block sympathetic nerve activity transiently). In some cases, an implantable electrode device can be used to deliver electrical therapy signals to particular nerves in a manner that reduces or blocks efferent sympathetic nerve activity. Examples of electrode devices that can be used to deliver electrical therapy signals to particular nerves in a manner that reduces or blocks sympathetic nerve activity include, without limitation, those devices configured to include one or more electrodes as described elsewhere (see, e.g., U.S. Pat. No. 8,010,204, U.S. Pat. No. 7,783,362, U.S. Pat. No. 6,928,320, PCT Publication No. WO/2011/156439, U.S. Patent Application Publication No. 20040172085, U.S. Patent Application Publication No. 20050131485, U.S. Patent Application Publication No. 20070073354, U.S. Patent Application Publication No. 20100191311, U.S. Patent Application Publication No. 20120016448, U.S. Patent Application Publication No. 20100114244, or EP1904160). In some cases, neurostimulator devices such as a Medtronic™ Soletra or Kinetra can be implanted into a mammal and configured to deliver electrical therapy signals to particular nerves in a manner that reduces or blocks sympathetic nerve activity. For example, renal denervation, carotid sinus stimulation, and carotid body denervation or inhibition with drugs can be used to reduce efferent sympathetic traffic.

In some cases, an implantable drug pump device can be used to deliver one or more nerve blocking agents to particular nerves in a manner that reduces or blocks sympathetic nerve activity. Examples of nerve blocking agents that can be used to reduce or block sympathetic nerve activity as described herein include, without limitation, ethanol or phenol. Any appropriate drug pump device can be used to deliver one or more nerve blocking agents to a location described herein to reduce or block sympathetic nerve activity. Examples of such drug pump devices that can be configured to deliver one or more nerve blocking agents to a location described herein included, without limitation, those drug pump devices described in U.S. Pat. Nos. 7,226,442; 7,648,677; 8,012,119; 5,527,307; International Patent Application Publication No. WO2000/074753, or U.S. Patent Application Publication No. 2007/0275035.

Any appropriate dose of a nerve blocking agent can be delivered as described herein provided that that amount reduces or blocks sympathetic nerve activity. For example, ethanol (e.g., pure ethanol) can be delivered to a nerve to reduce or block nerve activity.

The invention will be further described in the following examples, which do not limit the scope of the invention described in the claims.

### EXAMPLES

### Example 1

### Aging Enhances Autonomic Support of Blood Pressure in Women

The effect of ganglionic blockade on arterial blood pressure and how this relates to baseline muscle sympathetic nerve activity in 12 young (25±1 years) and 12 older postmenopausal (61±2 years) women were examined. The women were studied before and during autonomic blockade using trimethaphan camsylate. At baseline, muscle sympathetic nerve activity burst frequency and burst incidence were higher in the older women (33±3 versus 15±1 bursts/min; 57±5 versus 25±2 bursts/100 heartbeats, respectively; P<0.05). Muscle sympathetic nerve activity bursts were abolished by trimethaphan within minutes (e.g., between about 6 and about 35 minutes). Older women had a greater decrease in mean arterial pressure (−29±2 versus −9±2 mm Hg; P<0.01) and total peripheral resistance (−10±1 versus −5±1 mm Hg/L per minute; P<0.01) during trimethaphan. Baseline muscle sympathetic nerve activity was associated with the decrease in mean arterial pressure during trimethaphan (r=−0.74; P<0.05). See, also, Barnes et al., Hypertension, 63:303-308 (2014).

In summary, these results demonstrate that autonomic support of blood pressure is greater in older women compared with young women and that elevated sympathetic nerve activity in older women contributes importantly to the increased incidence of hypertension after menopause.

### Example 2

### Distinguishing High and Low Sympathetic Activity Individuals

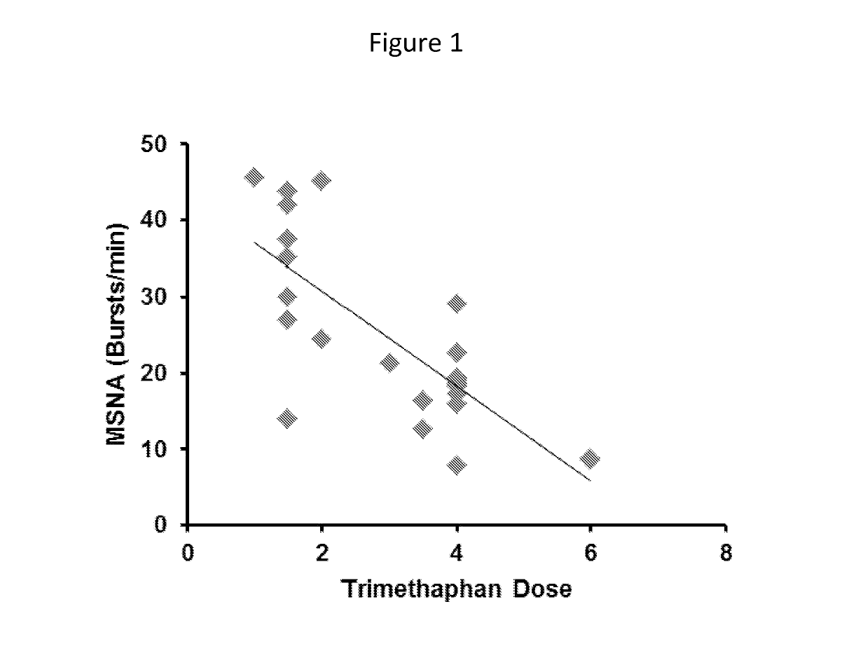

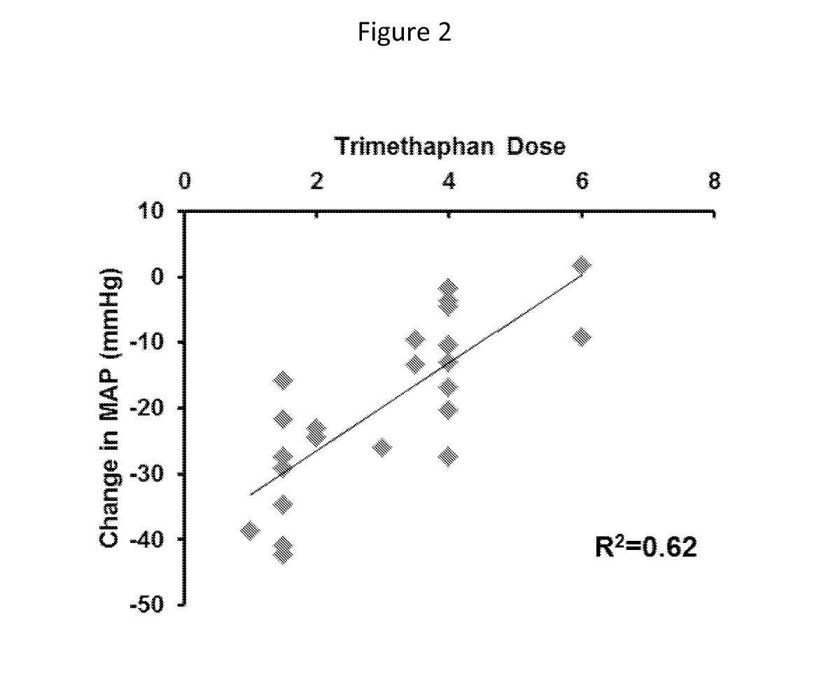

Both normal human subjects and patients with diseases such as resistant hypertension can have very wide ranges of baseline sympathetic activity. The following was developed to distinguish those individuals with high levels of baseline sympathetic activity from individuals with lower levels of baseline sympathetic activity. Trimethaphan camsylate, a ganglionic blocking drug, was administered (e.g., by infusion) at 1-4 mg/minute for 5-10 minutes to 24 healthy humans, while non-invasive beat-to-beat measurements of arterial pressure were made. A fall in blood pressure during brief escalating doses of trimethaphan camsylate was directly related to baseline sympathetic activity in healthy humans. Individuals with high levels of sympathetic activity exhibited larger reductions in blood pressure. The relationship between the fall in blood pressure and baseline sympathetic activity in a group of about 20-30 healthy women ranging in age from their early 20's to their later 60's was determined (FIGS. 1 and 2). Similar data were obtained using healthy men. These results demonstrate that measurements of blood pressure (e.g., arterial pressure), sympathetic activity, or both during or following administration of a ganglionic blocking drug can be used to distinguish high and low sympathetic activity individuals. Those individuals with an elevated level of sympathetic activity responsive to a ganglionic blocking drug (e.g., a reduction in the number of bursts per minute that is greater than 10 burst per minute) can be optimal candidates for sympatholytic therapy.

### Example 3

### Treating Resistant Hypertension

A person suffering from resistant hypertension is administered a ganglionic blocking drug (e.g., trimethaphan camsylate) or another sympatholytic agent or combination of sympatholytic agents (e.g., phentolamine to block alpha-adrenergic receptors followed by esmolol to block beta-adrenergic receptors) by, for example, infusion. When administering phentolamine followed by esmolol, between a loading dose of 0.15 mg/kg of phentolamine is used followed by a maintenance dose of 0.015 mg/kg followed by between about 25 and 300 mg of esmolol per minute for about 5 to 10 minutes. Blood pressure measurements (e.g., beat-to-beat measurements of arterial pressure) are obtained for about 5 to 10 minutes prior to, during, and for about 5 to 10 minutes after administration of the sympatholytic agents. From these measurements, the degree of blood pressure drop in response to the administrations is determined A blood pressure drop greater than 20 mmHg indicates that the person has high sympathetic activity and is to be treated using a sympatholytic therapy. A blood pressure drop that is less than 20 mmHg indicates that the person has low sympathetic activity and is not to be treated using a sympatholytic therapy. In some cases, sympathetic activity is measured in addition to blood pressure or in place of blood pressure to determine if the person has high or low sympathetic activity.

After the person is identified as having high sympathetic activity, the person is subjected to a sympatholytic therapy such as a neuroablation technique. In some cases, the person is treated for the resistant hypertension by renal denervation, carotid sinus nerve stimulation, or carotid body denervation.

### Example 4

### Treating Congestive Heart Failure

A person suffering from congestive heart failure is administered a ganglionic blocking drug (e.g., trimethaphan camsylate) or another sympatholytic agent or combination of sympatholytic agents (e.g., phentolamine to block alpha-adrenergic receptors followed by esmolol to block beta-adrenergic receptors) by, for example, infusion. When administering trimethaphan camsylate, between about 0.5 mg and 10 mg of trimethaphan camsylate is administered per minute (e.g., about 1 to 4 mg per minute) for about 2 to 20 minutes (e.g., 5 to 10 minutes). When administering phentolamine followed by esmolol, between a loading dose of 0.15 mg/kg of phentolamine is used followed by a maintenance dose of 0.015 mg/kg followed by between about 25 and 300 mg of esmolol per minute for about 5 to 10 minutes. Blood pressure measurements (e.g., beat-to-beat measurements of arterial pressure) are obtained for about 5 to 10 minutes prior to, during, and for about 5 to 10 minutes after administration of the sympatholytic agents. From these measurements, the degree of blood pressure drop in response to the administrations is determined A blood pressure drop greater than 20 mmHg indicates that the person has high sympathetic activity and is to be treated using a sympatholytic therapy. A blood pressure drop that is less than 20 mmHg indicates that the person has low sympathetic activity and is not to be treated using a sympatholytic therapy. In some cases, sympathetic activity is measured in addition to blood pressure or in place of blood pressure to determine if the person has high or low sympathetic activity.

After the person is identified as having high sympathetic activity, the person is subjected to a sympatholytic therapy such as a neuroablation technique, a therapy that includes implanting an implantable device designed to deliver a sympatholytic therapy, or both. In some cases, the person is treated for congestive heart failure by renal denervation, carotid sinus nerve stimulation, or carotid body denervation.

### Example 5

### Treating Chronic Kidney Disease

A person suffering from chronic kidney disease is administered a ganglionic blocking drug (e.g., trimethaphan camsylate) or another sympatholytic agent or combination of sympatholytic agents (e.g., phentolamine to block alpha-adrenergic receptors followed by esmolol to block beta-adrenergic receptors) by, for example, infusion. When administering trimethaphan camsylate, between about 0.5 mg and 10 mg of trimethaphan camsylate is administered per minute (e.g., about 1 to 4 mg per minute) for about 2 to 20 minutes (e.g., 5 to 10 minutes). When administering phentolamine followed by esmolol, between a loading dose of 0.15 mg/kg of phentolamine is used followed by a maintenance dose of 0.015 mg/kg followed by between about 25 and 300 mg of esmolol per minute for about 5 to 10 minutes. Blood pressure measurements (e.g., beat-to-beat measurements of arterial pressure) are obtained for about 5 to 10 minutes prior to, during, and for about 5 to 10 minutes after administration of the sympatholytic agents. From these measurements, the degree of blood pressure drop in response to the administrations is determined A blood pressure drop greater than 20 mmHg indicates that the person has high sympathetic activity and is to be treated using a sympatholytic therapy. A blood pressure drop that is less than 20 mmHg indicates that the person has low sympathetic activity and is not to be treated using a sympatholytic therapy. In some cases, sympathetic activity is measured in addition to blood pressure or in place of blood pressure to determine if the person has high or low sympathetic activity.

After the person is identified as having high sympathetic activity, the person is subjected to a sympatholytic therapy such as a neuroablation technique, a therapy that includes implanting an implantable device designed to deliver a sympatholytic therapy, or both. In some cases, the person is treated for chronic kidney disease by renal denervation, carotid sinus nerve stimulation, or carotid body denervation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating hypertension, wherein said method comprises:
(a) administering one or more sympatholytic agents to a mammal having hypertension,
(b) detecting a greater than 20 mmHg reduction in blood pressure after administration of said one or more sympatholytic agents to said mammal, and
(c) ablating or stimulating an efferent or afferent nerve within said mammal or implanting a pump configured to deliver a sympatholytic agent to said efferent or afferent nerve,
wherein a symptom of said hypertension is reduced following said step (c).

2. The method of claim 1, wherein said mammal is a human.

3. The method of claim 1, wherein said hypertension is resistant hypertension.

4. The method of claim 1, wherein said step (a) comprises administering trimethaphan camsylate as said one or more sympatholytic agents.

5. The method of claim 1, wherein said step (a) comprises administering phentolamine and esmolol as said one or more sympatholytic agents.

6. The method of claim 1, wherein said step (c) comprises ablating said efferent or afferent nerve within said mammal.

7. The method of claim 1, wherein said step (c) comprises implanting said pump configured to deliver said sympatholytic agent to said efferent or afferent nerve.

8. The method of claim 1, wherein said method comprises ablating a renal nerve.

9. The method of claim 1, wherein said method comprises ablating a renal nerve by applying radiofrequency ablation to said renal nerve.

10. A method for treating heart failure, wherein said method comprises:
(a) administering one or more sympatholytic agents to a mammal with heart failure,
(b) detecting a greater than 20 mmHg reduction in blood pressure after administration of said one or more sympatholytic agents to said mammal, and
(c) ablating or stimulating an efferent or afferent nerve within said mammal or implanting a pump configured to deliver a sympatholytic agent to said efferent or afferent nerve,
wherein a symptom of said heart failure is reduced following said step (c).

11. The method of claim 10, wherein said mammal is a human.

12. The method of claim 10, wherein said heart failure is congestive heart failure.

13. The method of claim 10, wherein said step (a) comprises administering trimethaphan camsylate as said one or more sympatholytic agents.

14. The method of claim 10, wherein said step (a) comprises administering phentolamine and esmolol as said one or more sympatholytic agents.

15. The method of claim 10, wherein said step (c) comprises ablating said efferent or afferent nerve within said mammal.

16. The method of claim 10, wherein said step (c) comprises implanting said pump configured to deliver said sympatholytic agent to said efferent or afferent nerve.

17. The method of claim 10, wherein said method comprises ablating a renal nerve.

18. The method of claim 10, wherein said method comprises ablating a renal nerve by applying radiofrequency ablation to said renal nerve.

19. A method for treating kidney disease, wherein said method comprises:
(a) administering one or more sympatholytic agents to a mammal with kidney disease,
(b) detecting a greater than 20 mmHg reduction in blood pressure after administration of said one or more sympatholytic agents to said mammal, and
(c) ablating or stimulating an efferent or afferent nerve within said mammal or implanting a pump configured to deliver a sympatholytic agent to said efferent or afferent nerve,
wherein a symptom of said kidney disease is reduced following said step (c).

20. The method of claim 19, wherein said mammal is a human.

21. The method of claim 19, wherein said kidney disease is chronic kidney disease.

22. The method of claim 19, wherein said step (a) comprises administering trimethaphan camsylate as said one or more sympatholytic agents.

23. The method of claim 19, wherein said step (a) comprises administering phentolamine and esmolol as said one or more sympatholytic agents.

24. The method of claim 19, wherein said step (c) comprises ablating said efferent or afferent nerve within said mammal.

25. The method of claim 19, wherein said step (c) comprises implanting said pump configured to deliver said sympatholytic agent to said efferent or afferent nerve.

26. The method of claim 19, wherein said method comprises ablating a renal nerve.

27. The method of claim 19, wherein said method comprises ablating a renal nerve by applying radiofrequency ablation to said renal nerve.
